(19) United States
(12) Patent Application Publication
(10) Pub. No.: US 2009/0234456 A1
(43) Pub. Date: Sep. 17, 2009

(54) Intervertebral Implant and Methods of Implantation and Treatment

(75) Inventor: Jeffrey H. Nycz, Collierville, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/048,627

(22) Filed: Mar. 14, 2008

(51) Int. Cl. A61F 2/44

(52) U.S. Cl. 623/17.16; 623/17.15

(57) ABSTRACT

Intervertebral implants and associated methods of implantation and treatment are provided. In one aspect, an intervertebral implant for positioning between an upper vertebra and a lower vertebra is provided. The implant includes an upper endplate, a lower endplate, and at least one support having a variable stiffness positioned between the upper and lower endplates. In some instances, the implant includes a sensing element for monitoring a characteristic of the intervertebral implant, a processor for determining a desired stiffness of the supports based on the characteristic monitored by the sensing element, and an actuator for adjusting the stiffness of the supports to the desired stiffness. In another aspect, a prosthetic device for a spinal joint is provided. In yet another aspect, a method of treating a motion segment of a vertebral column is provided.

### TECHNICAL FIELD

Embodiments of the present disclosure relate generally to intervertebral implants and associated methods of implantation and treatment.

### BACKGROUND

Within the spine, the intervertebral disc functions to stabilize and distribute forces between vertebral bodies. It comprises a nucleus pulposus which is surrounded and confined by the annulus fibrosis.

Intervertebral discs are prone to injury and degeneration. For example, herniated discs typically occur when normal wear or exceptional strain causes a disc to rupture. Degenerative disc disease typically results from the normal aging process, in which the tissue gradually loses its natural water and elasticity, causing the degenerated disc to shrink and possibly rupture. Intervertebral disc injuries and degeneration may be treated by fusion of adjacent vertebral bodies or by replacing the intervertebral disc with an implant, also known as a prosthesis or prosthetic device. Generally, fusion of the adjacent vertebral bodies prevents movement between the adjacent vertebrae and the range of motion provided by the natural intervertebral disc. Some implants, on the other hand, preserve at least some of the range of motion provided by the natural intervertebral disc.

Although existing devices and methods associated within intervertebral implants have been generally adequate for their intended purposes, they have not been entirely satisfactory in all respects. The devices and methods in this disclosure overcome one or more of the shortcomings of the prior art.

### SUMMARY

In one aspect, a spinal implant is provided.

In a further aspect, an intervertebral implant for positioning between an upper vertebra and a lower vertebra is provided. The intervertebral implant comprises an upper endplate for engaging the upper vertebra and a lower endplate for engaging the lower vertebra. At least one support having a variable stiffness is positioned between the upper endplate and the lower endplate. The intervertebral implant also includes at least one sensing element for monitoring a characteristic of the intervertebral implant. A processor is in communication with the at least one sensing element for determining a desired stiffness of the at least one support based on the characteristic monitored by the sensing element. An actuator in communication with the processor adjusts the stiffness of the at least one support to the desired stiffness based on a signal received from the processor.

In some instances, the supports include a magnetorheological fluid. In that regard, in some instances the actuator controls a viscosity of the magnetorheological fluid of the supports. In some embodiments, the actuator includes a power supply for producing an electromagnetic field through the magnetorheological fluid to control the viscosity. In some instances, the characteristic monitored by the at least one sensing element is a load, acceleration, or rotation related to the intervertebral implant. In some instances, the supports include a temperature-activated metal alloy. In one particular embodiment, the temperature-activated metal is Nitinol. Accordingly, in some embodiments the actuator delivers an electric current to the temperature-activated metal to control the stiffness of the supports. In some instances, the processor continuously determines the desired stiffness of the supports and the actuator continuously adjusts the stiffness of the supports to the desired stiffness. In that regard, in some embodiments the actuator adjusts the stiffness of the supports to the desired stiffness within 10 ms of receiving the signal from the processor.

In a further aspect, a prosthetic device for treating a spinal joint is provided. The prosthetic device comprises a first endplate having a first engagement surface and a second endplate having a second engagement surface. A plurality of supports having a variable stiffness are positioned between the upper endplate and the lower endplate. The prosthetic device also includes at least one sensor for monitoring a characteristic of the prosthetic device, at least one processor in communication with the at least one sensor for determining a desired stiffness for each of the plurality of supports based on a value of the characteristic monitored by the at least one sensor, and at least one actuator for adjusting the variable stiffness of each of the plurality of supports to the desired stiffness based on a signal received from the processor.

In some instances, the sensors, processors, and/or actuators are positioned at least partially within the endplates of the prosthetic device. In some embodiments, the actuator comprises a power supply that is in communication with the at least one sensor and the at least one processor. In one particular embodiment, the power supply is a rechargeable battery. In some instances, at least one of the plurality of supports includes a magnetorheological fluid and the at least one actuator produces an electromagnetic field to control a viscosity of the magnetorheological fluid. In that regard, in some embodiments the actuator produces a magnetic field to control the viscosity of the magnetorheological fluid. In some instances, the characteristic monitored by the at least one sensor is a load on the prosthetic device. In that regard, in some embodiments the desired stiffnesses for the supports as determined the processor are inversely proportional to the load on the prosthetic device as measured by the sensors. In some embodiments, at least one of the plurality of supports comprises a temperature-activated metal and the actuator controls a voltage supplied to the temperature-activated metal to control the stiffness of the support.

In another aspect, a method of treating a motion segment of a vertebral column is provided. The method includes providing a self-adjusting intervertebral implant and implanting the self-adjusting intervertebral implant within the motion segment of the vertebral column. In some instances the implant includes at least one support having a variable stiffness, at least one sensing element for monitoring a load on the intervertebral implant, a processor for determining a desired stiffness for the at least one support based on the load on the intervertebral implant, and an actuator in communication with the processor for adjusting the stiffness of the supports to the desired stiffness.

Additional aspects and features of the present disclosure will be apparent from the detailed description and claims as set forth below.

### BRIEF DESCRIPTION OF THE DRAWINGS

FIG. 1 is a diagrammatic side elevation view of an adult human vertebral column.

FIG. 2 is a diagrammatic side elevation view of a portion of the vertebral column of FIG. 1, depicting an intervertebral implant according to one aspect of the present disclosure positioned between two adjacent vertebrae.

FIG. 3 is a diagrammatic perspective view of the intervertebral implant of FIG. 2.

FIG. 4 is a diagrammatic partial cutaway perspective view of the intervertebral implant of FIGS. 2 and 3.

FIG. 5 is a diagrammatic front view of the intervertebral implant of FIGS. 2-4.

FIG. 6 is a diagrammatic partial cutaway rear view of the intervertebral implant of FIGS. 2-5.

FIG. 7 is a diagrammatic partial cutaway of the intervertebral implant similar to that of FIG. 6, but showing a lateral side view of the intervertebral implant.

FIG. 8 is a diagrammatic lateral side view of the intervertebral implant of FIGS. 2-7

FIG. 9 is a diagrammatic partial cutaway top view of the intervertebral implant of FIGS. 2-8.

FIG. 10 is a diagrammatic bottom view of the intervertebral implant of FIGS. 2-9.

FIG. 11 is a diagrammatic partial cutaway phantom perspective view of the intervertebral implant of FIGS. 2-10.

### DETAILED DESCRIPTION

For the purpose of promoting an understanding of the principles of the present disclosure, reference is made to the specific embodiments illustrated in the drawings, and specific language is used to describe the embodiments. It is nevertheless understood that no limitation of the scope of the present disclosure is intended. Any alterations and further modifications of the described embodiments, and any further applications of the principles of the present disclosure as described herein, are fully contemplated, as would occur to one skilled in the art to which the invention relates.

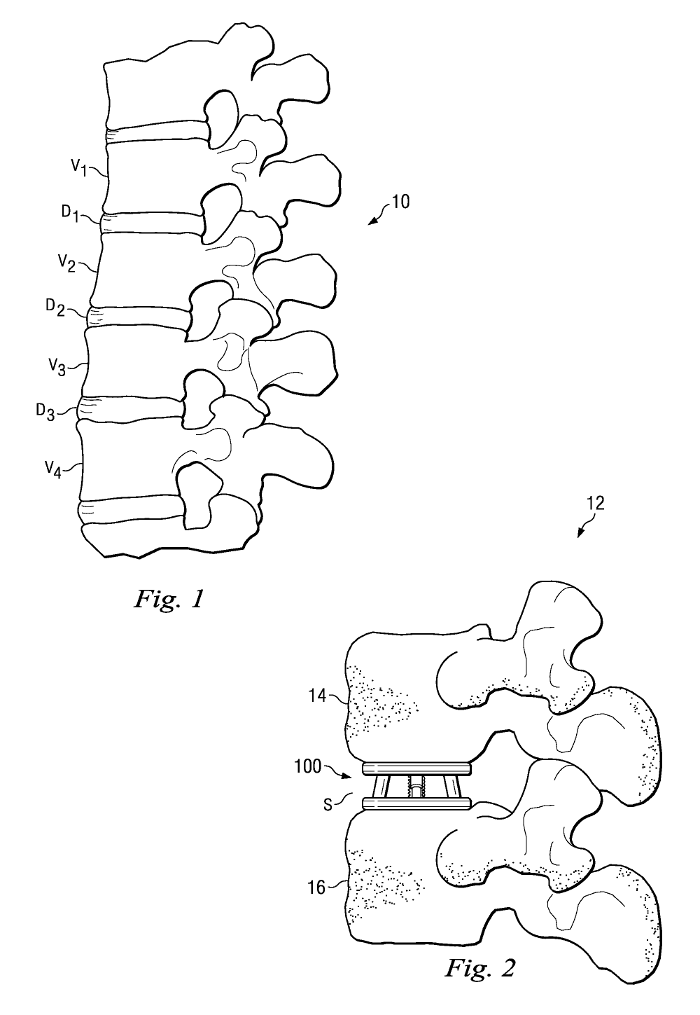

FIG. 1 illustrates a lateral view of a portion of a spinal column 10, illustrating a group of adjacent upper and lower vertebrae V1, V2, V3, V4 separated by natural intervertebral discs D1, D2, D3. Although the illustration generally depicts the lumbar region, it is understood that the devices, systems, and methods of this disclosure also may be applied to all regions of the vertebral column, including the cervical and thoracic regions. A vertebral joint comprises two adjacent vertebrae separated by an intervertebral disc. FIG. 2 illustrates an exemplary vertebral joint 12 including an upper vertebra 14 and a lower vertebra 16. In this illustration, instead of a natural intervertebral disc, an intervertebral implant 100 is disposed in the disc space S between the upper and lower vertebrae 14, 16 created by removal of the natural disc.

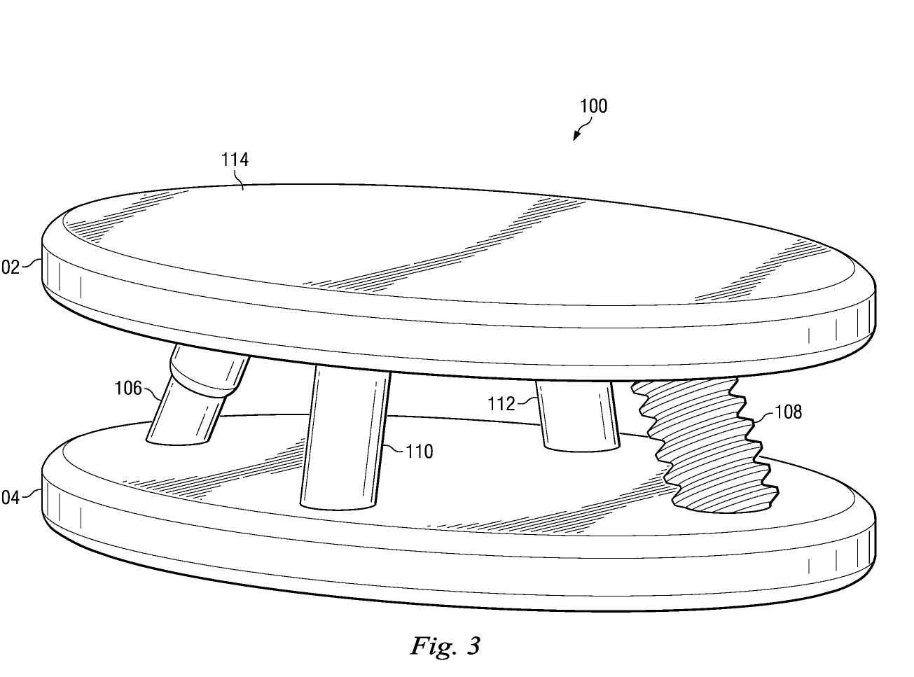

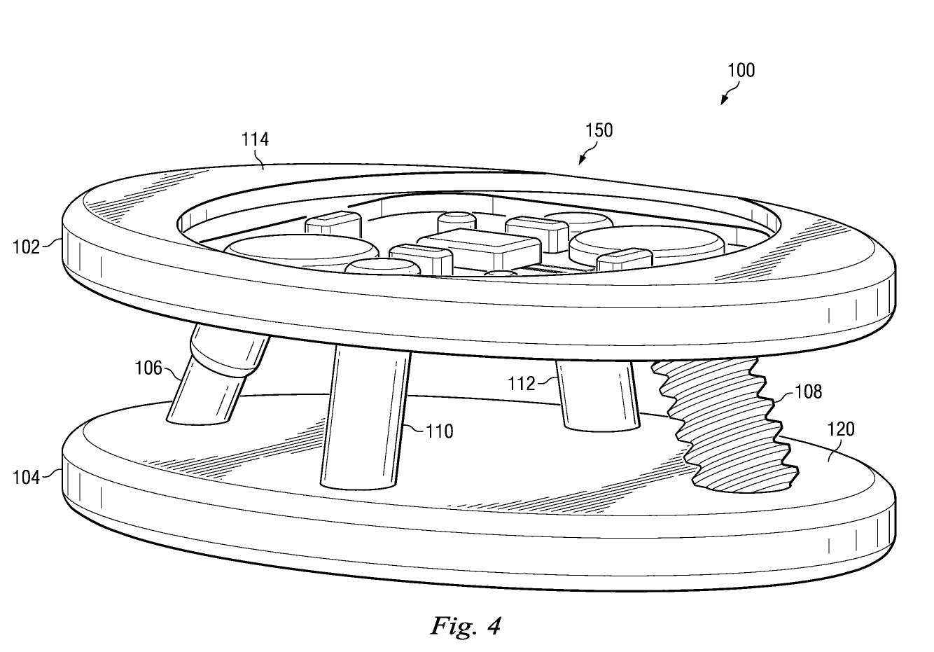

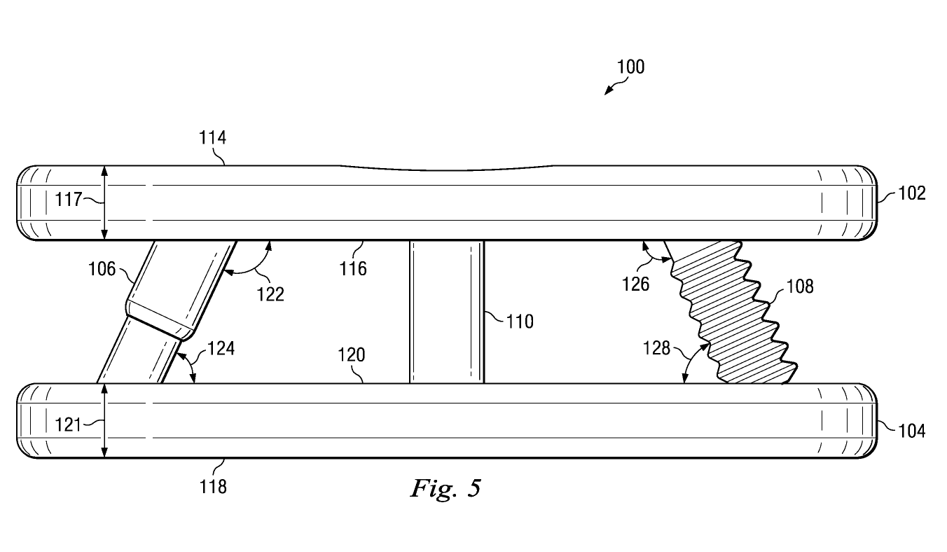

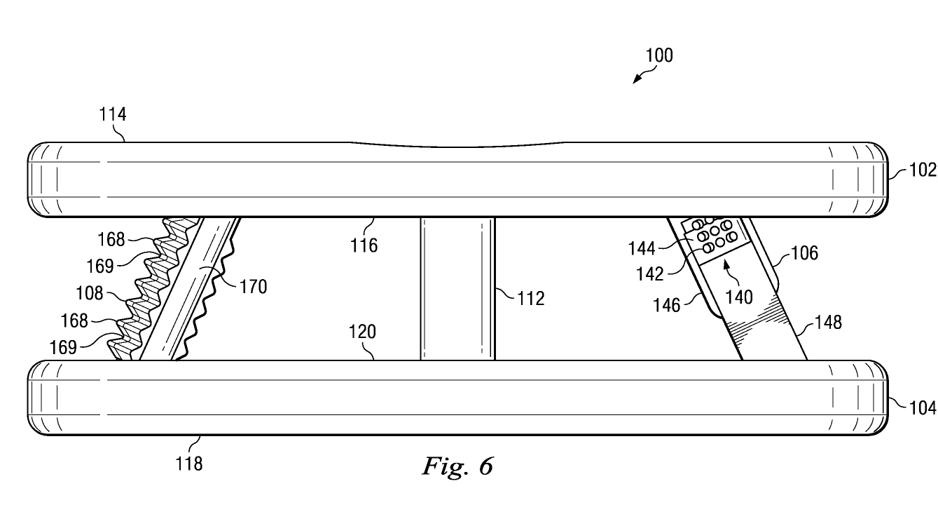

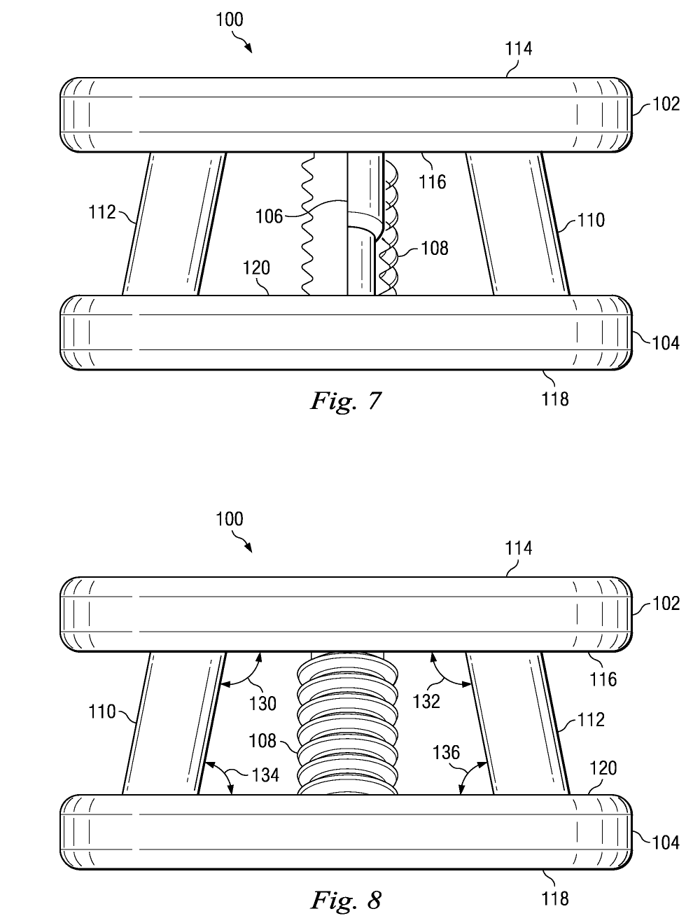

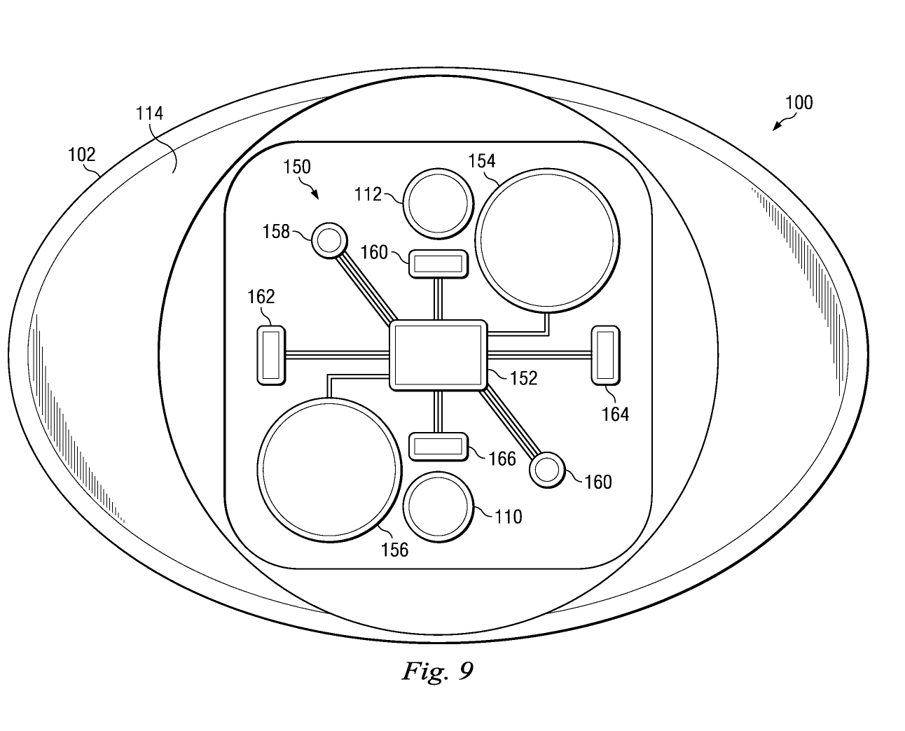

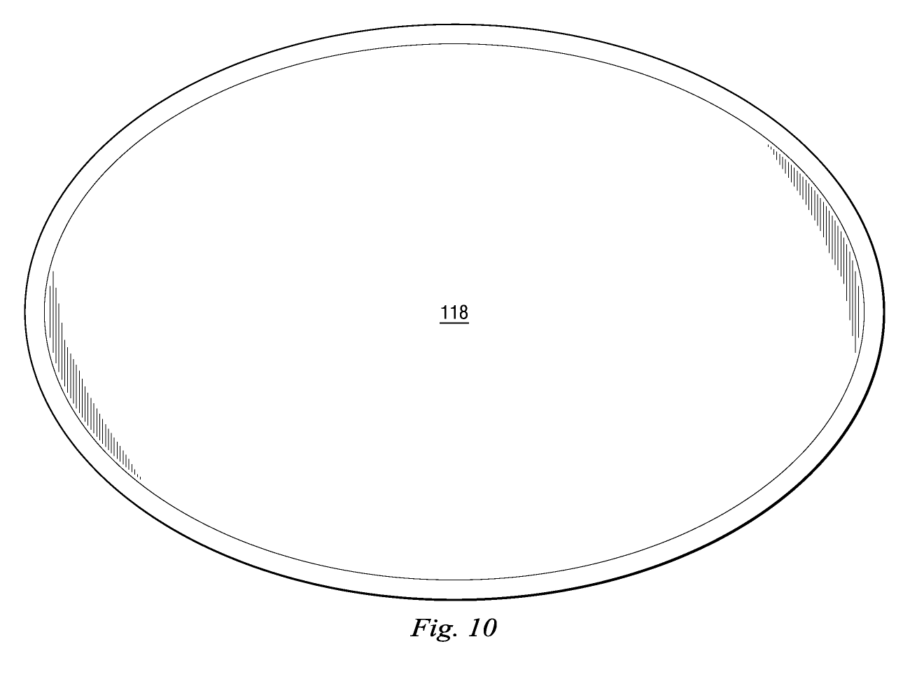

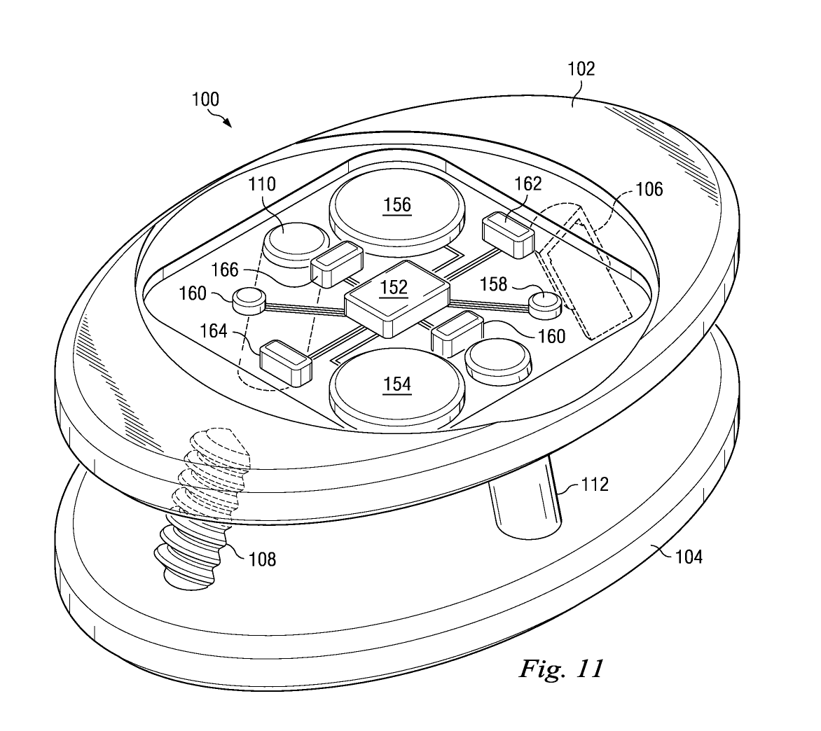

Referring generally to FIGS. 2-11, the intervertebral implant 100 will be discussed in greater detail. FIG. 2 is a diagrammatic side elevation view of the intervertebral implant 100 positioned between two adjacent vertebrae 14, 16 of a vertebral joint 12. FIG. 3 is a diagrammatic perspective view of the intervertebral implant 100. FIG. 4 is a diagrammatic perspective view of the intervertebral implant 100 similar to that of FIG. 3, but with partial cutaway to illustrate additional aspects of the intervertebral implant 100. FIG. 5 is a diagrammatic front view of the intervertebral implant 100. FIG. 6 is a diagrammatic partial cutaway rear view of the intervertebral implant 100. FIG. 7 is a diagrammatic partial cutaway of the intervertebral implant 100 similar to that of FIG. 6, but showing a lateral side view of the intervertebral implant. FIG. 8 is a diagrammatic lateral side view of the intervertebral implant 100 from the direction substantially opposite to that of FIG. 7. FIG. 9 is a diagrammatic partial cutaway top view of the intervertebral implant 100. FIG. 10 is a diagrammatic bottom view of the intervertebral implant 100. FIG. 11 is a diagrammatic partial cutaway phantom perspective view of the intervertebral implant 100.

Generally, the intervertebral implant 100 is sized to fit within the disc space S in a manner similar to that of a natural intervertebral disc, as shown in FIG. 2. The intervertebral implant 100 provides support and stabilization to the vertebrae 14, 16. In addition, the intervertebral implant 100 allows the upper vertebra 14 to move relative to the lower vertebra 16 to preserve at least some movement in the vertebral joint 12. In some instances, the intervertebral implant 100 has a variable stiffness to control the amount or degree of movement between the vertebrae 14, 16 and/or the amount of support provided to the vertebral joint 12. Further, in some embodiments the intervertebral implant 100 continuously self-adjusts its stiffness as necessary to maintain a desired amount of motion and support to the vertebral joint 12. In some embodiments, the stiffness of the intervertebral implant 100 is adjusted in the sagittal, axial, and/or coronal planes to provide the desired amount of motion and/or support.

Referring more specifically to FIG. 3, in the illustrated embodiment the intervertebral implant 100 includes an upper endplate 102, a lower endplate 104, and four supports 106, 108, 110, and 112 extending between the upper and lower endplates. The upper endplate 102 is configured to engage the upper vertebra 14 in some instances. To that end, the upper endplate 102 includes an engagement surface 114 for engaging the endplate of the upper vertebra 14. As discussed in greater detail below, the engagement surface 114 includes various features for enhancing the engagement with the upper vertebra in some embodiments. Referring to FIG. 5, opposite the engagement surface 114, the upper endplate 102 includes an inner surface 116. In the present embodiment the inner surface 116 is substantially planar and the supports 106, 108, 110, and 112 extend from the inner surface 116 towards the lower endplate 104. The upper endplate 102 has a thickness 117 between the engagement surface 114 and the inner surface 116. In some embodiments, the thickness 117 of the upper endplate 102 is sufficient to allow one or more electronic components of the intervertebral implant to be positioned therein. In some instances, the thickness 117 is between about 2 mm and about 25 mm, and in some instances between about 5 mm and about 15 mm.

Similarly, the lower endplate 104 is configured to engage the lower vertebra 16 in some instances. To that end, the lower endplate 104 includes an engagement surface 118, best viewed in FIG. 10, for engaging the endplate of the lower vertebra 16. In some embodiments, the engagement surface 118 also includes various features for enhancing the engagement with the lower vertebra. Referring again to FIG. 5, opposite the engagement surface 118, the lower endplate 104 includes an inner surface 120. In the illustrated embodiment, the inner surface 120 is substantially planar and the supports 106, 108, 110, and 112 extend from the inner surface 120 towards the upper endplate 102. The lower endplate 104 has a thickness 121 between the engagement surface 118 and the inner surface 120. In some embodiments, the thickness 121 of the lower endplate 104 is sufficient to allow one or more electronic components of the intervertebral implant to be positioned therein. In some instances, the thickness 121 is between about 2 mm and about 25 mm, and in some instances between about 5 mm and about 15 mm.

Referring to FIGS. 3-8, the supports 106, 108, 110, and 112 extending between the upper endplate 102 and the lower endplate 104 control the amount of cushioning or support provided by the intervertebral implant 100 and/or the amount of motion allowed by the implant. In some instances, the supports 106, 108, 110, and 112 allow the intervertebral implant 100 to compress or elastically deform under compressive loads. Further, in some instances the supports 106, 108, 110, and 112 allow the intervertebral implant 100 to expand or elastically stretch in response to a force that pulls the endplates 102, 104 away from one another. Further, in some instances, the supports 106, 108, 110, and 112 allow both compression and expansion of the intervertebral implant. In some instances, a portion of the intervertebral implant 100 is compressed while another portion of the intervertebral implant is expanded. For example, when positioned in the cervical spine, a posterior portion of the implant 100 may be compressed during extension of the vertebral joint while an anterior portion of the implant is expanded. As another example, in a lateral bending to the patient's right side, the lateral right side of the implant 100 may be compressed while the lateral left side of the implant is expanded or stretched.

In some embodiments of the present disclosure, the supports 106, 108, 110, and 112 continually adjust the amount of the support and/or motion of the implant 100 based on parameters associated with the patient's physical activity. For example, in some instances the supports 106, 108, 110, and 112 adjust the based on an anatomical load imparted on the implant. In other embodiments, the adjustment is based on acceleration, motion, pressure, and/or other parameters associated with the vertebral joint. In some instances, the stiffness of the supports 106, 108, 110, and 112 is adjusted in order to adjust the support provided by the implant and/or the motion allowed by the implant. In that regard, the stiffness of the supports 106, 108, 110, and 112 is adjusted in a different manner depending on the type of support utilized. In accordance with the present disclosure support 106 and support 108 represent two different types of support and each will be described in greater detail below. The supports 110 and 112 are representative of a generic support and, therefore, will not be discussed in great detail below. In some embodiments, the supports 110 and 112 are substantially similar to the support 106 and/or the support 108. In that regard, in some instances the implant 100 includes all of the same type of supports. In other instances, the implant includes at least two different types of support in accordance with the present disclosure. In some instances, the implant combines one or more of the supports of the present disclosure with previously known implant features and/or supports.

Referring more particularly to FIG. 6, the support 106 generally comprises a shock absorber having a magnetorheological fluid 140 disposed therein. The magnetorheological fluid 140 has variable viscosity. In particular, the viscosity of the magnetorheological fluid 140 varies based on the strength of the magnetic field applied to the fluid. The greater the magnetic field applied to the magnetorheological fluid 140, the greater the viscosity of the fluid. As illustrated, the magnetorheological fluid 140 comprises a plurality of ferrous particles 142 suspended in a carrier fluid 144. In some instances, the carrier fluid 144 is a silicon based fluid and the ferrous particles are iron particulate. Under the presence of a magnetic field the ferrous particles 142 are polarized and form a chain-like formation within the carrier fluid 144. Generally the ferrous particles 142 form along the direction of the magnetic flux passing through the magnetorheological material 140, such that the strength of the ferrous particle chain is directly related to the strength of the magnetic field.

As shown, the magnetorheological material 140 is positioned within a cylinder 146 of the support 106 that mates with a rod 148. In some embodiments, the cylinder 146 is substantially fixed with respect to the upper endplate 102 and the rod 148 is substantially fixed with respect to the lower endplate 104. In that regard, the cylinder 146 is partially imbedded within the upper endplate 102 in the present embodiment. In some instances, the cylinder 146 is integrally formed with the upper endplate 102. Similarly, in some embodiments the rod 148 is partially imbedded within the lower endplate 104 and is integrally formed with the lower endplate in some instances.

Together, the cylinder 146, rod 148, and magnetorheological fluid 140 act as a shock absorber for the implant 100. By adjusting the viscosity of magnetorheological fluid 140 the stiffness of the support 106, and in turn the implant 100, is adjusted. Thus, in some embodiments the implant 100 includes electronics 150, as seen in FIG. 4 for example and discussed in greater detail below, for controlling and producing a magnetic field for adjusting the viscosity of the magnetorheological fluid 140. In some instances, the electronics 150 produce an electric current in a portion of the upper end plate 102 and/or cylinder 146 that generates a corresponding magnetic field through the magnetorheological fluid 140. In that regard, in some instances the electronics 150 determine the appropriate amount of electric current to be provided to achieve a desired viscosity at least partially based on an attribute associated with the patient's activity. For example, in some instances the electric current is determined by the electronics based on a load on the implant, an acceleration of a portion of the implant, and/or a pressure on the implant. The viscosity of the magnetorheological fluid 140 is capable of changing within a few milliseconds (generally less than 10 milliseconds) of being subjected to the magnetic field generated by the electric current from the electronics. Accordingly, in some embodiments the implant 100 is capable of approximately real time adjustment of the stiffness of the support 106 based on the patient's physical activities and/or attributes associated with the patient's activity.

Referring more particularly to FIGS. 4 and 9, the electronics 150 include a processor 152 (“processor” is understood to include microprocessors) that is connected to a pair of power supplies 154 and 156, a pair of load sensors 158 and 160, and four microelectromechanical systems (“MEMS”) devices 160, 162, 164, and 166. As illustrated in FIG. 4, the electronics 150 are all positioned within the upper endplate 102 in the present embodiment. In that regard, as illustrated by FIG. 3, in some instances the electronics 150 are entirely enclosed within the upper endplate 102. In other embodiments, however, one or more of the electronic components 150 are positioned outside of the upper endplate 102 and/or co-planar with an outer surface of the upper endplate. While the electronics 150 are shown as being disposed entirely within the upper endplate 102, in other embodiments the electronics 150 are positioned partially or entirely within the lower endplate 104. Further, in some instances electronics are positioned in both the upper and lower endplates 102, 104. In that regard, in one particular embodiment the electronics may be hard-wire connected through a wire extending through the support 108.

In some embodiments, the processor 152 receives signals from the load sensors 158 and 160 and/or the MEMS devices 160, 162, 164, and 166 and determines the amount of voltage or current necessary to produce a magnetic field to adjust the viscosity of the magnetorheological fluid 140 to a desired level. Based on the processor's 152 determination, the appropriate amount of current is provided from one or more of the power supplies 154, 156. In some instances, the processor 152 continually monitors the signals received from the load sensors 158 and 160 and/or the MEMS devices 160, 162, 164, and 166 and continually dictates the appropriate current to be provided by the power supplies 154, 156 such that the support 106 provides the appropriate amount of stiffness at all times. In that regard, in some particular aspects the stiffness of the support 106 is adjusted within 10 ms of the processor 152 requesting a change in the stiffness. Also, in some embodiments the processor 152 is configured to associate data from the load sensors 158 and 160 and/or the MEMS devices 160, 162, 164, and 166 with typical activities of the patient, such as walking, sitting, standing, running, laying down, kneeling, and/or other activities. Based on the associated activity as determined by the processor, a corresponding current is generated to incite the appropriate amount of stiffness in the support 106.

As mentioned, the processor 152 determines the appropriate amount of stiffness for the support 106 and the corresponding amount of current based on signals received from the load sensors 158 and 160 and/or the MEMS devices 160, 162, 164, and 166. The load sensors 158, 160 monitor forces on the implant 100 resulting from loads on the vertebral joint and relay the corresponding loading information to the processor 152. The MEMS devices 160, 162, 164, and 166 monitor aspects of the implant and/or vertebral joint such as accelerations, rotations, and/or other motions. In that regard, the MEMS devices 160, 162, 164, and 166 send the resulting data to the processor 152 for consideration. While two load sensors and four MEMS devices have been disclosed, in other embodiments other combinations of load sensors and/or MEMS devices are utilized including only load sensors or only MEMS devices. Further still, in some instances only a single sensing element (load sensor or MEMS device) is utilized.

In order to save battery power and/or computing power, in some instances signals from the load sensors 158 and 160 and/or the MEMS devices 160, 162, 164, and 166 are conditioned or filtered before being sent to the processor 152. For example, in some instances a sufficient change in the force as measured by the load cells must occur before a signal is sent to the processor. In other instances, a threshold level of acceleration must be detected by the MEMS devices before a signal is sent to the processor. In this manner, the processor 152 may only be utilized when a change sufficient to trigger a change in the stiffness of the support 106 has been detected. In other instances, the load sensors 158 and 160 and/or the MEMS devices 160, 162, 164, and 166 continuously send all information to the processor 152 for consideration.

Supplying the power requirements for the processor 152 and the implant 100 in general are the power supplies 154 and 156. In that regard, the power supplies 152 and 154 generate the electrical currents that induce the magnetic fields that change the viscosity of the magnetorheological material 140 in some instances. In other instances, the power supplies 152 and 154 generate the electrical currents that change the material properties of the support 108 as discussed below. In some embodiments, the power source supplies 154, 156 are batteries. In this manner the electronics 150 may be internally powered. The batteries are lithium iodine batteries similar to those used for other medical implant devices such as pacemakers in some instances. It is understood that the battery may be any type of battery suitable for implantation. In some instances the battery is rechargeable. In that regard, in some specific embodiments the battery may be recharged by an external device so as to avoid the necessity of a surgical procedure to recharge the battery. For example, in one embodiment the battery is rechargeable via inductive coupling.

It is also contemplated that at least some of the electronic components be self-powered and not require a separate stored-energy power supply. For example, in some embodiments the load sensors 158, 160 and/or the MEMS devices 160, 162, 164, and 166 are piezoelectric such that signals detected by these components or other signals provide power to the sensor. In other embodiments, the electronic components utilize energy harvesting to recharge the power supplies 154, 156 or store energy for use by the electronic components. Energy harvesting in this context is understood to be energy generated by the patient's motion or natural body that is captured by the implant 100 for use in powering the electronics. Additional and/or alternative sources of power may be utilized in other embodiments. In that regard, while two power supplies are illustrated, it is understood that in other embodiments a single power supply or a greater number of power supplies may be utilized.

Referring more particularly to FIG. 6, the support 108 is generally shaped like a bellow. In that regard, the support 108 includes a plurality of alternating protrusions 168 and recesses 169 on its outer surface and a generally cylindrical core 170. The bellow shape of the support 108 allows it to be flexible in some instances. In that regard, the support 108 is formed of a temperature-activated metal alloy in some embodiments. The mechanical properties of the metal alloy are dependent upon its temperature. In some instances, the metal alloy becomes stiffer as its temperature increases. One specific example of such an alloy is Nitinol. Accordingly, in some embodiments the stiffness of the support 108 increases as the temperature of the support increases. In some embodiments, a current is introduced into the support 108 to control the temperature of the metal alloy. Generally, an increase in the current results in an increase in the stiffness of the support 108. Similarly, a decrease in the current passing through the support reduces the stiffness of the support 108. Accordingly, by controlling the amount of current passing through the support 108, the stiffness of the support and, in turn, the implant is controlled. Thus, in some embodiments the electronics 150 of the implant 100 are utilized in a substantially similar manner as that described above with respect to the support 106. For example, the processor 152 determines the current to be applied to the support 108 based on a desired stiffness for the support. In some instances, this determination is based on a physical activity and/or a measurable attribute associated with the patient's physical activity. In this manner, the support 108 is utilized to provide continuous adjustment of the stiffness of the support 108 based on the patient's physical activities and/or attributes associated with the patient's activity. In some embodiments, the inner core or inner surface of the support 108 is not cylindrical. In one particular embodiment, the inner portion of the support 108 includes a plurality of alternating protrusions and recesses corresponding to the protrusions 168 and recesses 169 of the outer surface.

In some embodiments, the supports 106, 108, 110, and 112 allow the implant 100 to facilitate motion in vertebral joint 12, including but not limited to flexion, extension, lateral bending, rotation, and translation between the vertebrae 14, 16. Accordingly, in some instances the supports 106, 108, 110, and 112 allow corresponding motion between the endplates 102 and 104. Thus, in some embodiments, supports 106, 108, 110, and 112 allow the endplates 102, 104 to translate with respect to one another in the anterior-posterior direction, the lateral directions, and/or the inferior-superior direction. Further, in some embodiments the supports 106, 108, 110, and 112 allow the endplates 102, 104 to rotate with respect to one another. In other embodiments, the supports 106, 108, 110, and 112 limit motion of the implant 100 and, therefore, the vertebral joint 12 in one or more directions. In that regard, the supports 106, 108, 110, and 112 entirely prevent or limit movement in a particular direction and/or permit movement in the particular direction up to a certain level. Accordingly, in some embodiments the supports 106, 108, 110, and 112 include a hard stop that prevents movement beyond the desired amount.

In some instances, the maximum amount or range of movement allowed by the supports 106, 108, 110, and 112 is varied over time. For example, in some instances it is desirable to further limit motion of the implant 100 in one or more directions and/or provide more rigid support to the vertebral joint over time due to the patient's physical conditions or other factors. In other instances, it is desirable to allow greater range of motion in one or more directions and/or provide less rigid support to the vertebral joint over time or after a set period of time after implantation. As discussed above, the amount of movement or range of motion allowed by the supports 106, 108, 110, and 112 is determined by a processor or other control mechanism in some embodiments. Accordingly, in some instances the processor or other control mechanism is reprogrammed or otherwise configured to facilitate the change in the range of motion of the device.

In the present embodiment, the supports 106, 108, 110, and 112 are equally spaced about the outer portion of the prosthetic device. In particular, the supports 106 and 108 are positioned on the lateral portions of the implant 100, while the supports 110 and 112 are positioned on the anterior and posterior portions of the implant. Further, each of the supports 106, 108, 110, and 112 extend generally at an oblique angle with respect to the inner surfaces 116 and 120 of the upper and lower endplates 102, 104. For example, referring more specifically to FIG. 5, as shown therein the support 106 extends at an angle 122 from the inner surface 116 of the upper endplate 102 and interfaces the inner surface 120 of the lower endplate 104 at an angle 124. In the present embodiment, the angles 122 and 124 are supplementary angles. However, in some embodiments, the angles 122 and 124 are not supplementary. Generally, each of the angles 122 and 124 is between about 30 degrees and about 150 degrees. In some rare embodiments the angles 122, 124 may be outside of these ranges. Accordingly, in some instances the support 106 engages the inner surface 116 of the upper endplate 102 at a position closer to a midpoint of the implant than the position where the support engages the inner surface 120 of the lower endplate 104. Further, in some instances the support 106 extends substantially perpendicular to both the upper endplate 102 and the lower endplate 104.

Similarly, the support 108 extends at an angle 126 from the inner surface 116 of the upper endplate 102 and extends at an angle 128 from the inner surface 120 of the lower endplate 104. The angles 126 and 128 are supplementary angles in the present embodiment, but are not supplementary in all embodiments. Generally, each of the angles 126 and 128 is between about 30 degrees and about 150 degrees. In some rare embodiments the angles 126, 128 may be outside of these ranges. Accordingly, similar to the support 106, in some instances the support 108 engages the inner surface 116 of the upper endplate 102 at a position closer to a midpoint of the implant than the position where the support 108 engages the inner surface 120 of the lower endplate 104. Further, in some instances the support 108 extends substantially perpendicular to the inner surfaces 116, 120 of both the upper endplate 102 and the lower endplate 104.

Referring to FIG. 8, the supports 110 and 112 also extend at angles 130 and 132 from the inner surface 116 of the upper endplate 102, respectively. The supports 110 and 112 extend at angles 134 and 136 from the inner surface 120 of the lower endplate 104, respectively. Similar to the supports 106 and 108 above, angles 130, 132 and angles 134, 136 are supplementary angles, respectively, in the present embodiment. However, in other embodiments the angles may not be supplementary. Generally, each of the angles 130, 132, 134, and 136 is between about 30 degrees and about 150 degrees. In some rare embodiments the angles 130, 132, 134, and 136 may be outside of these ranges. Accordingly, in some instances one or both of the supports 110 and 112 engage the inner surface 116 of the upper endplate 102 at a position closer to a midpoint of the implant than the position where the support 110 or 112 engages the inner surface 120 of the lower endplate 104. Further, in some instances one or both of the supports 110 and 112 extend substantially perpendicular to the inner surfaces 116, 120 of both the upper endplate 102 and the lower endplate 104.

While the present embodiment of the intervertebral implant 100 includes the four supports 106, 108, 110, and 112, in other embodiments the intervertebral implant includes a greater or less number of supports. In that regard, in some instances the intervertebral implant includes three supports. In one such embodiment, two of the supports are positioned adjacent a posterior portion of the device and equally spaced laterally from a midline of the implant, while the third support positioned adjacent an anterior portion of the device and substantially centered on the midline. In another embodiment, two of the supports are positioned adjacent an anterior portion of the device and equally spaced laterally from a midline of the implant, while the third support positioned adjacent a posterior portion of the device and substantially centered on the midline. Generally, any number of supports may be utilized.

Further, in some embodiments multiple implants 100 may be implanted into a single patient at multiple levels of the spine. In some embodiments, the multiple implants 100 form a networked implant system. For example, in some instances the effect of stiffening a support of one of the implants is considered at each level where an implant is positioned. By taking into account more than one level of the spine where an implant is positioned instead of only the single level of a particular implant, a global support for the spinal column is established. In this manner, the networked implants prevent the implants from working against one another. In such situations, the implants 100 communicate with one another via wireless telemetry or other suitable means. In some instances, each of the implants communicates with a remote device that then controls the corresponding attributes of each of the implants. Accordingly, in some instances a patient begins with only a single implant, but then is implanted with another implant later. In such situations, the initial implant may not be configured for communication with the new implant, but would be configured for communication with the remote device such that the old and new implants could be networked together. In that regard, it is contemplated that the remote device may be programmable and/or backwards compatible to communicate with implants previously implanted.

In some instances, the implant includes memory for storing performance data for the implant. In that regard, the implant may also include a wireless telemetry component so that the stored data may be communicated to an external receiver. In some instances, the external receiver also includes a memory unit. In that regard, the memory unit of the external receiver may be adapted for multiple uses. First, the memory unit may be adapted for permanent storage of the performance data obtained from the implant. Thus, the memory unit may store data obtained at various times from the implant so the data may later be reviewed, compared, or analyzed. Second, the memory unit may be adapted for temporary storage of performance data obtained from the implant. In this case, the memory unit will store the data until it is either discarded or transferred for permanent storage. For example, the data may be transferred from the memory unit of the external receiver via a networking interface to a network or computer for permanent storage. In some instances, such a networking interface provides a means for the external receiver to communicate with other external devices. The type of network utilized may include such communication means as telephone networks, computer networks, or any other means of communicating data electronically.

In some instances, the networking interface of the external receiver could obviate the need for the patient to even go into the doctor's office for obtaining implant performance data. For example, the patient could utilize an external receiver to obtain the usage data from the implant on a scheduled basis (e.g. daily, weekly, monthly, etc.). Then, utilizing the networking interface the patient could send this data to the treating medical personnel. The networking interface may be configured to directly access a communication network such as a telephone or computer network for transferring the data. It is fully contemplated that the computer network be accessible by treating medical personnel for reviewing implant performance data of the patient without requiring the patient to make an actual visit to the doctor's office. In some instances, the networking interface is similar to the CareLink system from Medtronic, Inc.

It is also contemplated that any communication between the external receiver and the computer network may be encrypted or otherwise secured so as protect the patient's privacy. It is also contemplated that the networking interface may be configured for communication with a separate device that is adapted for accessing the communication network. For example, the networking interface may be a USB connection. The external receiver may be connected to a personal computer via the USB connection and then the personal computer may be utilized to connect to the communication network, such as the internet, for transferring the data to a designated place where the treating doctor may receive it.

Further, supports in accordance with the present disclosure may be utilized in combination with other spinal implant motion preserving features. For example, in some instances one or more supports are utilized in combination with a ball-and-socket articulating joint of the implant. In one embodiment, the one or more supports are positioned around the ball-and-socket joint. In that regard, the supports are utilized to control the movement and/or support provided to the vertebral joint by the implant. In some instances, the supports are utilized to limit movement of the implant in a particular direction or orientation.

The upper and lower endplates assemblies 102, 104 are formed of suitable biocompatible materials. In instances, metals such as cobalt-chromium alloys, titanium alloys, nickel titanium alloys, and/or stainless steel alloys are utilized. Ceramic materials such as aluminum oxide or alumnia, zirconium oxide or zirconia, compact of particulate diamond, and/or pyrolytic carbon are suitable in some instances. In some embodiments, polymer materials are utilized, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE.

Further, the exterior engagement surfaces 114, 118 of the upper and lower endplates 102, 104 include features or coatings (not shown) that enhance the fixation of the implanted prosthesis in some embodiments. For example, the surfaces 114, 118 are roughened such as by chemical etching, bead-blasting, sanding, grinding, serrating, and/or diamond-cutting in some instances. All or a portion of the exterior surfaces 28, 34 may also be coated with a biocompatible and osteoconductive material such as hydroxyapatite (HA), tricalcium phosphate (TCP), and/or calcium carbonate to promote bone in growth and fixation. Alternatively, osteoinductive coatings, such as proteins from transforming growth factor (TGF) beta superfamily, or bone-morphogenic proteins, such as BMP2 or BMP7, may be used. Other suitable features may include spikes, ridges, keels, fins, posts, or other bone engaging protrusions for initial fixation of the intervertebral implant 100 and/or to prevent migration in the lateral or anterior/posterior directions. In some instances, the exterior surfaces 114, 118 include serrations, diamond cuts, and/or other surface textures.

In the illustrated embodiment of FIGS. 2-11, the implant 100 includes elliptical, oval, or oblong endplates 102, 104 as viewed from the top or bottom of the intervertebral implant (FIGS. 9 and 10 for example). In other embodiments, the endplates have other shapes, including rectangular, rectangular with curved sides, kidney shaped, heart shaped, square, oval, triangular, hexagonal, or any other shape suitable for mating with the vertebrae 12, 14. Further, in the illustrated embodiment of FIGS. 2-11, the engagement surfaces 114, 118 extend relatively parallel to one another. However, in other embodiments, the surfaces 114, 118 are angled with respect to each other to accommodate a desired lordotic or kyphotic angle. In that regard, the specific lordotic or kyphotic angle may be selected based on the level of spine in which the implant 100 is to be inserted. In some instances, the outer profile of the implant 100 is tapered, angled, or wedge shaped to create the desired lordotic or kyphotic angle. In some embodiments, the lordotic and kyphotic angles are created by utilizing one or more angled, tapered, or wedge shaped endplate assemblies. In that regard, the thickness of the endplate may vary along the length and/or width of the prosthetic device to achieve the angled orientation.

In other embodiments, the endplates 102, 104 have substantially planar surfaces 114, 118 and substantially constant thicknesses, but are positioned at a lordotic or kyphotic angle due to the orientation of the supports positioned between the endplates. In some instances, the supports have a neutral position that positions the endplates in a lordotic or kyphotic angle. In some instances, the supports are biased towards the lordotic or kyphotic neutral position. In some embodiments, the relative heights of the supports are controlled by a processor and/or actuator to achieve the desired lordotic or kyphotic angle. In that regard, the processor and/or actuator will direct one or more of the supports to have an increased or decreased height relative to one or more other supports.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” and “right,” are for illustrative purposes only and can be varied within the scope of the disclosure. In the claims, means-plus-function clauses are intended to cover the structures described herein as performing the recited function and not only structural equivalents, but also equivalent structures.

What is claimed:

1. An intervertebral implant for positioning between an upper vertebra and a lower vertebra, the implant comprising:
an upper endplate for engaging the upper vertebra;
a lower endplate for engaging the lower vertebra;
at least one support having a variable stiffness positioned between the upper endplate and the lower endplate;
at least one sensing element for monitoring a characteristic of the intervertebral implant;
a processor in communication with the at least one sensing element for determining a desired stiffness of the at least one support based on the characteristic monitored by the sensing element; and
an actuator in communication with the processor for adjusting the stiffness of the at least one support to the desired stiffness based on a signal received from the processor.

2. The intervertebral implant of claim 1, wherein the at least one support comprises a magnetorheological fluid.

3. The intervertebral implant of claim 2, wherein the actuator controls a viscosity of the magnetorheological fluid.

4. The intervertebral implant of claim 3, wherein the actuator comprises a power supply for producing an electromagnetic field through the magnetorheological fluid.

5. The intervertebral implant of claim 1, wherein the characteristic monitored by the at least one sensing element is a load on the intervertebral implant.

6. The intervertebral implant of claim 1, wherein the characteristic monitored by the at least one sensing element is an acceleration.

7. The intervertebral implant of claim 1, wherein the characteristic monitored by the at least one sensing element is a rotation of a portion of the intervertebral implant.

8. The intervertebral implant of claim 1, wherein the at least one support comprises a temperature-activated metal.

9. The intervertebral implant of claim 8, wherein the temperature-activated metal comprises Nitinol.

10. The intervertebral implant of claim 8, wherein the actuator delivers an electric current to the temperature-activated metal.

11. The intervertebral implant of claim 8, wherein the actuator comprises a power supply.

12. The intervertebral implant of claim 1, wherein the processor continuously determines the desired stiffness of the at least one support and the actuator continuously adjusts the stiffness of the at least one support to the desired stiffness based on the signal received from the processor.

13. The intervertebral implant of claim 12, wherein the actuator adjusts the stiffness of the at least one support to the desired stiffness within 10 ms of receiving the signal from the processor.

14. A prosthetic device for a spinal joint, comprising:
a first endplate having a first engagement surface;
a second endplate having a second engagement surface;
a plurality of supports having a variable stiffness positioned between the first endplate and the second endplate;
at least one sensor for monitoring a characteristic of the prosthetic device;
at least one processor in communication with the at least one sensor for determining a desired stiffness for each of the plurality of supports based on a value of the characteristic monitored by the at least one sensor; and
at least one actuator in communication with the at least one processor for adjusting the variable stiffness of each of the plurality of supports to the desired stiffness based on a signal received from the processor.

15. The prosthetic device of claim 14, wherein the at least one sensor, at least one processor, and at least one actuator are positioned at least partially within the first endplate.

16. The prosthetic device of claim 14, wherein the at least one actuator comprises a power supply in communication with the at least one sensor and the at least one processor.

17. The prosthetic device of claim 14, wherein at least one of the plurality of supports comprises a magnetorheological fluid and wherein the at least one actuator produces an electromagnetic field to control a viscosity of the magnetorheological fluid.

18. The prosthetic device of claim 14, wherein the characteristic monitored by the at least one sensor is a load on the prosthetic device, and wherein the desired stiffness for each of the plurality of supports determined by the processor are inversely proportional to the load on the prosthetic device.

19. The prosthetic device of claim 14, wherein at least one of the plurality of supports comprises a temperature-activated metal and wherein the at least one actuator controls a voltage supplied to the temperature-activated metal.

20. A spinal implant, comprising:
a first endplate having a first engagement surface for engaging a first vertebra;
a second endplate having a second engagement surface for engaging a second vertebra;
a plurality of supports having a variable stiffness positioned between the first endplate and the second endplate, each of the plurality of supports comprising a magnetorheological fluid;
at least one sensor for monitoring a load on the prosthetic device; and
at least one actuator for adjusting a viscosity of the magnetorheological fluid based on the load on the prosthetic device.
